(19) United States
(12) Patent Application Publication
(10) Pub. No.: US 2004/0259834 A1
(43) Pub. Date: Dec. 23, 2004

(54) Therapeutic composition containing at least diflomotecan and capecitabine

(76) Inventors: Philip G. Kasprzyk, Boston, MA (US); Gregoire Prevost, Antony (FR); Paola Principe-Nicolas, Gif-Sur-Yvette (FR)

Correspondence Address: MUSERLIAN, LUCAS AND MERCANTI, LLP, 475 PARK AVENUE SOUTH, 15TH FLOOR, NEW YORK, NY, 10016, US

(21) Appl. No.: 10/465,309

(22) Filed: Jun. 18, 2003

(51) Int. Cl. A61K 31/7072

(52) U.S. Cl. 514/50

(57) ABSTRACT

The method of treating cancer by administering to a patient in need thereof a synergistic effective amount of a composition comprising at least a first component of diflomotecan and a second component of capecitabine.

The present invention is directed to a therapeutic composition comprising at least diflomotecan in synergistic combination with capecitabine. This composition is particularly useful for the treatment of cancers.

The invention relates to the treatment of cancer with therapeutic synergistic combinations of diflomotecan and capecitabine and the use of such combinations for an improved treatment.

It was unexpectedly found that administration of the two components in accordance with the present invention results in improved antineoplastic effects that are significantly superior to the results obtained with each compound alone. Namely, administration of the two components in accordance with the present invention resulted in an improved therapeutic index (that is, superior efficacy) in comparison to either component alone without a significant increase in toxicity. Alternatively, the invention permits reduction of the amount of at least one component (in comparison the amount typically given in monotherapy) while retaining a desirable therapeutic index. In preferred embodiments, the amount of both components (in comparison to the amount typically given in monotherapy) is reduced affording reduced toxicity while still retaining a desirable therapeutic index.

Chemotherapy and more particularly combined chemotherapy is one of the means well accepted to fight cancers. Thus the combination of different antitumor agents may be a way to increase the antitumoral efficacy when a synergistic effect is revealed and/or less toxicity is observed.

Diflomotecan (BN80915) is an E-ring modified camptothecin derivative. This novel topoisomerase I inhibitor bears a novel 7-membered β-hydroxylactone ring structure characterized by a more stable lactone form. As a result it showed a greater plasma stability which potentially can enhance anti-tumor activity. It has previously been evaluated against various xenografts growing in nude mice and has demonstrated excellent activity (Prevost et al—AACR 93^{rd} Annual Meeting—Apr. 6-10, 2002, San Francisco, Calif.; Salazar et al—ASCO 2001 Annual Meeting—May 12-15, 2001, San Francisco, Calif.; Larsen et al—Cancer Research 61, 2961-2967, Apr. 1, 2001; Prevost et al—AACR 92^{nd} Annual Meeting—Mar. 24-28, 2001—New Orleans, La.; Gelderblom et al—AACR 92^{nd} Annual Meeting—Mar. 24-28, 2001—New Orleans, La.; Kasprzyk et al—AACR 92^{nd} Annual Meeting—Mar. 24-28, 2001—New Orleans, La.; Lansiaux et al—Molecular Pharmacology, 60:450-461, 2001; Demarquay et al—Anti-Cancer Drugs 2001, 12, pp. 9-19; Urasaki et al—Cancer Research 60, 6577-6580, Dec. 1, 2000; Lavergne et al—The camptothecins, volume 922 of the Annals of the New York Academy of Sciences, December 2000; Salazar et al—11th NCI-EORTC-AACR Symposium on New Drugs in Cancer Therapy—Amsterdam—Nov. 7-10, 2000; Bonneterre et al—11th NCI-EORTC-AACR Symposium on New Drugs in Cancer Therapy—Amsterdam—Nov. 7-10, 2000; Bailly et al—AACR 91^{st} Annual Meeting—Apr. 1-5, 2000.; Kasprzyk et al—AACR 91^{st} Annual Meeting—Apr. 1-5, 2000; Osheroff—Current Opinion in Oncologic, Endocrine & Metabolic Investigational Drugs 2000 2(3): 320-323.; Larsen et al—AACR-NCI-EORTC International Conference—Nov. 16-19, 1999; Principe et al—AACR-NCI-EORTC International Conference—Nov. 16-19, 1999; Menargues et al—AACR-NCI-EORTC International Conference—Nov. 16-19, 1999; Celma et al—AACR-NCI-EORTC International Conference—Nov. 16-19, 1999; Prunonosa et al—AACR-NCI-EORTC International Conference—Nov. 16-19, 1999; Kasprzyk et al—Annual Meeting of the AACR—Apr. 10-14, 1999).

Capecitabine (commercialy available as Xeloda®) is known to be useful in cancer therapeutics. Its main therapeutical indications are the treatment of colorectal and breast cancer.

The invention relates more particularly to a method of treating cancer, said method comprising administering to a patient in need thereof a synergistic effective amount of a therapeutic composition containing at least a first component consisting of diflomotecan, and a second component consisting of capecitabine.

According to the method of the present invention, the two components: diflomotecan and capecitabine may be administered simultaneously, separately or sequenced over time. When administered separately, diflomotecan may be administered first or capecitabine may be administered first.

A method according to the present invention is particularly suitable to treat breast cancer, colon cancer or rectum cancer.

The invention also relates more particularly to the use of diflomotecan in synergistic combination with capecitabine, for the preparation of a medicament intended to treat cancer, and more particularly to breast cancer, colon cancer or rectum cancer.

The present invention also relates to a product comprising a first component consisting of a therapeutic composition containing diflomotecan as active ingredient, and a second component consisting of a therapeutic composition containing capecitabine as active ingredient, as synergistic combination product for simultaneous or separate use, or use sequenced over time for treating cancer. More particularly, a product according to the present invention is suitable to treat breast cancer, colon cancer or rectum cancer.

A therapeutic composition of the present invention consists of the synergistic combination of diflomotecan and capecitabine in which each compound may be present in unseparate form and thus may be administered simultaneously, or in separate form and thus may be administered simultaneously or separately or sequenced over time. According to the present invention, the synergistic combination of diflomotecan and capecitabine enhances the antitumor activity of diflomotecan and of capecitabine and thus yields to a most effective and less toxic treatment of cancer.

The combination of diflomotecan and capecitabine may contain other additional components having a therapeutic activity. Thus, a composition of the present invention contains a combination of diflomotecan and capecitabine and optionally at least one additional component selected from the group consisting of topoisomerase I inhibitor, such as irinotecan or topotecan; topoisomerase II inhibitor; farnesyl transferase inhibitor; platinium derivatives such as cisplatin or carboplatin; antimetabolites such as 5-fluorouracil; alkylating agents such as cyclophosphamide, fosfamides or melphalan; antibiotics; hormonal agents.

The administration of said composition may also be associated with radiotherapy.

In a preferred embodiment, the topoisomerase I inhibitor as additional component may be a compound as defined in the PCT applications WO 97/00876, WO 98/28304, WO 98/28305, WO 99/11646 and WO 00/50427.

Therapeutic compositions as defined above can be utilized for the treatment of cancer and advantageously breast cancer, colon cancer and rectum cancer.

Therapeutic compositions as defined above comprise effective therapeutic quantities of at least diflomotecan and capecitabine, and pharmaceutically acceptable supports to form together or separately liquid composition(s) such as solutions or suspension, or solid composition(s) such as compressed tablets, pills, powders.

Irrespective of the use of the administration, i.e. simultaneously or separately use or sequenced over time, the two components: diflomotecan and capecitabine can be administered by identical or different administration routes. They can be administered by identical or different administration routes when they are present in separate form, and by identical administration routes when they are present in unseparate form. Diflomotecan can be administered by standard administration routes such as oral, intramuscular, intraperitoneal, sub-cutaneous or intravenous. Capecitabine can be also administered by standard administration routes and preferably by oral route. The other additional compounds may be administered by their recommended administration routes in the treatment of cancers. Advantageously, the administration of diflomotecan, capecitabine and the optional additional components is carried out according to a regimen dependent on the type of cancer and more particularly on the type of tumors.

The dosage ranges for the administration of the combination of diflomotecan and capecitabine according to the present invention may vary with the administration routes, as well as the state of the patient (age, extend of the disease). Diflomotecan can be administered at a daily dose comprised between 0.01 and 15 mg/m², preferably between 0.01 and 0.63 mg/m² by oral route.

Capecitabine which is a well known and marketed compound is administered at doses usually recommended in the treatment of cancer. Thus capecitabine is advantageously administered orally at a dose betweeen 800 and 3000 mg/m²/day, and preferably between 1250 and 2600 mg/m²/day.

Unless defined in another manner, all the technical and scientific terms used here have the same meaning as that commonly understood by an ordinary specialist in the field to which the invention belongs. Similarly, all publications, Patent Applications, all Patents and all other references mentioned here are incorporated by way of reference.

The following examples are presented to illustrate the above procedures and must in no way be considered as limiting the scope of the invention.

### EXPERIMENTAL PART

Methods

Mice: Female NCr-nude mice, 6-8 weeks of age, were fed ad libitum water (reverse osmosis, 0.17% Cl) and an autoclaved standard rodent (NIH31) diet consisting of: 18% protein; 5% fat; 5% fiber; 8% ash; and 3% minerals. Mice were housed in microisolators on a 12-hour light cycle at 22° C. (72° F.) and 40%-60% humidity.

Tumors : Mice in both models were implanted subcutaneously with 5×10⁶ HT-29 cells in the flank. Tumors were monitored initially twice weekly, and then daily as the neoplasms reached the desired size, approximately 100 mm³ (100 mg). When the colon carcinomas attained a size between 77-128 mg in calculated tumor weight, the animals were pair-matched into the various treatment groups (group mean tumor weights ranged from 98-100 mg). Estimated tumor weight was calculated using the formula:

Tumor weight (mg)=(w²×l)/2

where w=width and l=length in mm of a colon carcinoma.

Drugs:

Diflomotecan was prepared for oral administration according to following instructions. The vehicle was aqueous water containing 3% dimethyl-acetamide, 1.8% Montanox 80 and 0.2% NaCl. The solution of BN-80915 in vehicle was prepared fresh weekly for p.o. dosing. The dose range of diflomotecan is between 0.01 and 0.2 mg/kg.

Xeloda® (capecitabine; Roche) were each obtained as the marketed pharmaceutical drugs. The dose range of Xeloda® is between 100 mg/kg and 1000 mg/kg.

1. A method of treating cancer, comprising administering to a patient in need thereof a synergistic effective amount of at least a first component consisting of diflomotecan, and a second component consisting of capecitabine sufficient to treat cancer.

2. The method of claim 1, wherein diflomotecan and capecitabine are administered simultaneously.

3. The method of claim 1, wherein diflomotecan and capecitabine are administered separately.

4. The method of claim 3, wherein diflomotecan is administered first.

5. The method of claim 3, wherein capecitabine is administered first.

6. The method of claim 1, wherein diflomotecan and capecitabine e are administered sequenced over time.

7. The method of claim 1, wherein the cancer is selected from the group consisting of breast cancer, colon cancer and rectum cancer.

8-9. (cancelled)

10. A composition for treating cancer comprising a synergistic amount of a first component consisting of diflomotecan as active ingredient, and a second component consisting of capecitabine as active ingredient.

11-13. (cancelled)

14. A composition of claim 10, wherein the cancer is breast cancer, colon cancer or rectum cancer.
